(19) United States
(12) Patent Application Publication
(10) Pub. No.: US 2004/0162447 A1
(43) Pub. Date: Aug. 19, 2004

(54) 2-methyl-4-ethyl-2-octene-1-aldehyde

(76) Inventors: Thomas Markert, Monheim (DE); Ralph Nemitz, Juchen (DE); Marc Speitkamp, Dusseldorf (DE); Theo Ten Pierik, LE Venlo (NL); Markus Schneider, Duisburg (DE); Ralph Bunn, Krefeld (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/469,840

(22) PCT Filed: Feb. 26, 2002
(86) PCT No.: PCT/EP02/01990

(30) Foreign Application Priority Data
Mar. 7, 2001 (DE) 101 10 857.5

(51) Int. Cl. A61K 7/46; C07C 45/72

(52) U.S. Cl. 568/461; 512/27

(57) ABSTRACT

The invention relates to 2-methyl-4-ethyl-2-octene-1-aldehyde of formula (I), which is characterized by interesting and original odor characteristics which diffuse extremely well. The inventive substance is suitable for use as fragrance, for example in cosmetic preparations, technical products or in alcoholic perfumery.

### FIELD OF THE INVENTION

The present invention relates to 2-methyl-4-ethyl-2-octene-1-aldehyde, a method for the production thereof as well as the use thereof as fragrance.

### PRIOR ART

Many natural fragrances are not available, in view of their demand, in sufficient amounts. For example, 5000 kilos of rose blossoms are necessary to yield 1 kg rose oil. The consequence is a greatly limited annual production worldwide as well as a high price. It is therefore clear that the fragrance industry has a constant need for new fragrances with interesting scents. On the one hand the range of naturally available fragrances can be supplemented thereby and on the other hand it is possible thereby to be able to undertake the necessary adaptation to the ever changing fashion in taste. Moreover it becomes possible by this means to be able to cover the ever increasing demand for scent enhancers for products of daily use, such as cosmetics and cleaning agents.

Moreover there is generally a constant demand for synthetic fragrances which can be produced inexpensively and with a uniform high quality and have the original olfactory characteristics. In particular they should have a pleasant scent profile which is as natural as possible, qualitatively novel, of sufficient intensity and capable of advantageously influencing the scent of cosmetic goods and goods of daily use. In other words: There is a constant demand for compounds which have a characteristic novel scent profile and at the same time a high degree of adhesiveness, intensity of scent and strong diffusion.

DE-A-26 13 996 discloses reaction products of butanal with different aldehydes, with unsaturated condensation products resulting thereby. The reaction takes place with alkali hydroxide in the presence of a phase transfer catalyst.

### DESCRIPTION OF THE INVENTION

It was found that the compound of the general formula (I) excellently fulfills the above-mentioned requirements in every way and can be used in an advantageous manner as fragrance with shades of smell valuable from a perfume point of view and with good adhesiveness.

The subject matter of the present invention is, initially, 2-methyl-4-ethyl-2-octene-1-aldehyde of the general structure (I)

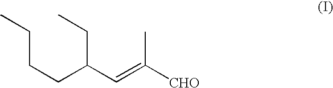

As formula (I) reveals, the C═C double bond is trans-configured as regards the long alkyl radical and the aldehyde group.

In a further embodiment, the invention relates to the use as fragrance of 2-methyl-4-ethyl-2-octene-1-aldehyde.

The compound (I) according to the invention is characterized by an odor characteristic in which iris and costus scents dominate. It has excellent stability in formulations for cosmetics and perfumeries of daily use.

The invention pertains further to a method for the production of compound (I) by reaction of 2-ethylhexanal with propionaldehyde in the presence of one or more organic compounds containing nitrogen as catalysts in an organic solvent.

Preferably such organic compounds containing nitrogen are used as catalysts which contain at least one positively charged N atom for each molecule, for example benzyltrimethyl ammonium hydroxide. Bases containing nitrogen are also suited as catalysts, such as 1,8-diazabicyclo[5.4.0]undec-7-en and similar compounds well known to the person skilled in the art. Preferably the reaction is carried out in the absence of alkali metal hydroxides.

Mixtures of organic solvents are to be considered also as “organic solvents”. Suitable solvents are, for instance, toluene, xylenes, cyclohexan, methylcyclohexan and the like. The reaction temperature is preferably adjusted in the range of 20 to 200° C. Especially preferred is the range from 50 to 150° C.

In perfume compositions compound (I) enhances ideally the harmony and the diffusion as well as the naturalness and also the adhesiveness, with the dosage being adjusted to each smell targeted, taking the other components of the composition into consideration.

That compound (I) has the above-mentioned scents was not predictable and is thus a further confirmation of the general experience that the olfactory characteristics of known fragrances do not allow automatic conclusions to be drawn regarding the properties of compounds having related structures since neither the perception mechanism of smells nor the influence of the chemical structure on the perception of smell has been sufficiently researched, and thus normally it cannot be predicted whether an altered structure of known fragrances will result at all in a change of the olfactory characteristics and whether these changes will be positively or negatively assessed by the person skilled in the art.

The compound of formula (I) is suited, due to its smell profile, in particular also for the modification and enhancement of known compositions. Their exceptional scent strength should in particular be emphasized, which contributes quite generally to the refinement of the composition.

The compound of formula (I) can be combined with numerous known fragrance ingredients, for example other fragrances of natural, synthetic or partial-synthetic origin, essential oils and plant extracts. The range of natural fragrances can include thereby not only high-volatile but also middle- and low-volatile components. The range of the synthetic fragrances can include representatives of practically all substance classes.

Examples for suitable substances with which the compound (I) can be combined are in particular:

(a) Natural products such as evernia furfuraceae absolute, basilicum oil, citrus oils such as bergamot oil, mandarin oil, etc., mastic absolute, myrtle oil, palmarosa oil, patchouli oil, petit grain oil, absinth oil, myrrh oil, olibanum oil, ceder wood oil, sandal wood oil, East Indian, guajak wood oil, cabreuva,

(b) Alcohols such as farnesol, geraniol, citronellol, linalool, nerol, phenylethyl alcohol, rhodinol, cinnamyl alcohol, Sandalore [3-methyl-5-(2.2.3-trimethylcyclopent-3-en-1-yl)pentan-2-ol], Sandela [3-ioscamphyl-(5)-cyclohexanol], Muguetanol,

(c) Aldehydes such as citral, Helional®, alpha-hexylcinnamylaldehyde, citronellal hydrate, Lilial® [p-tert.-butyl-α-methyldihydrocinnamaldehyd], methylnonylacetaldehyde,

(d) Ketones such as allyl ionone, α-ionone, β-ionone, Isoraldein, methyl ionone, noot katone, Calone, α-, β- and γ irone, Damascone,

(e) Esters such as allyl phenoxyacetate, benzylsalicylate, cinnamylpropionate, citronellyll acetate, decylacetate, dimethylbenzylcarbinylacetate, ethylacetoacetate, hexenylisobutyrate, linalylacetate, methyldihydrojasmonate, vetiverylacetate, cyclohexylsalicylate, isobornylisobutyrate, Evernyl,

(f) Lactones such as gamma-undecalacton, 1-oxaspiro[4.4]nonan-2-on, cylopentadecanolide, ethylenbrassylate,

(g) Ethers such as Herbavert, Ambroxan,

as well as various further components often used in the perfume industry such as musk and sandal wood fragrances, indole, p-menthane-8-thiol-3-on, methyleugenol and methylanthranilate.

Noteworthy is furthermore how the compound of structure (I) rounds off the scents of a wide range of known compositions and harmonizes these without, however, being dominant in an unpleasant manner.

The usable proportions of the compound (I) according to the invention in fragrance compositions range from approximately 1-70% by wt., based on the entire mixture. Compound (I) as well as compositions containing (I) can be used both to perfume cosmetic preparations such as lotions, creams, shampoos, soaps, ointments, powders, aerosols, toothpastes, mouthwash, deodorants as well as also in alcoholic perfumery (e.g. eau de cologne, eau de toilette, extracts). There is also the possibility to use these to perfume technical products such as detergents and cleaning agents, fabric softeners and textile treating agents. To perfume these various products, these are added to the compositions in an amount effective olfactorally, in particular in a concentration of 0.01 to 2% by wt., based on the entire product. These values do not, however, constitute restrictive limits since the experienced perfumer can still attain effects with even lesser concentrations or can construct novel complexes with even higher dosages.

### EXAMPLES

The following examples 1 and 2 show two different ways to produce 2-methyl-4-ethyl-2-octene-1-aldehyde (I). Example 1 demonstrates the method according to the invention, example 2 on the other hand a method known from DE-A-26 13 996. It is shown that according to the method according to Example 2 the compound (I) is obtained with a far lesser yield than according to the method according to the invention according to example 1.

### Example 1

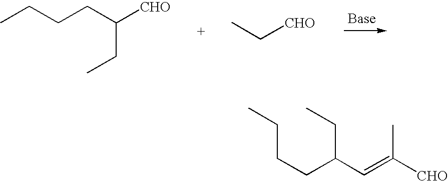

Charge:

1) 256,6 g (2 mol) 2-ethylhexanal

2) 124.9 g (0.3 mol) benzyltrimethyl ammonium hydroxide (40% solution in methanol)

3) 400 ml toluene

4) 232.0 g propionaldehyde

Apparatus: 2 L Agitator, drip funnel, reflux cooler, Pt 100.

Execution: The components 1), 2) and 3) were weighted out one after the other in nitrogen in the reaction flask while being stirred and heated to 83° C. reflux temperature. Component 4) was added continuously through the drip funnel drop by drop in 1.46 hours. Thereafter stirring continued for a further 2.5 hours at reflux temperature. The reaction rate control yielded a product content of 62.5 % with 5.6% educt.

Further Processing: The reaction mixture was washed neutral with water and ammonium chloride solution after being cooled to room temperature, was dried with sodium sulphate and concentrated on the rotation evaporator.

393.9 g raw product was distilled in a 20 cm Vigreux column. 172.1 g main product were obtained at boiling temperatures of 63-67° C./0.05 mbar and with a purity of 95.8% determined by gas chromatography.

Yield: 48.3% of the theoretical

Analysis: The IR spectrum (film between NaCl) showed characteristic shrinkage strands at 1641, 1690 (C═0) and 2708 cm⁻¹.

The ¹H-NMR (400 MHz in CDCl₃) showed signals for 2 methyl groups (2 superimposed triplets) at 0.9 ppm, and the methyl group on the double bond as singulet at 1.75 ppm. The 4 CH₂ groups yielded 2 signal groups, one at 1.25 ppm corresponds to 6 protons, the other at 1.55 ppm corresponds to 2 protons (broad multiplet). The proton at C-4 appeared as 2 superimposed quintets at 2.5 ppm. The olefinic proton resulted in a dublet at 6.2 ppm and the aldehyde proton appeared as singulet at 9.4 ppm.

Scent Characteristic: The first smell is of irises, costus; and the subsequent smell (after 24 hours on a scent strip) was balsamic, of myrrh resin, and a note of hair moss.

### Example 2

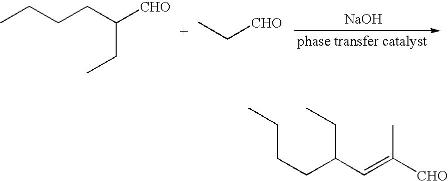

Charge:

1) 256.6 g (2 mol) 2 ethyl hexanal

2) 253.1 g (14.1 mol) demineralized water

3) 28.1 g (0.7 mol) sodium hydroxide

4) 18.9 g (47 mmol) tricaprylmethyl ammonium chloride

5) 400 ml toluene

6) 232.0 g (4.0 mol) propion aldehyde

Apparatus: 2 L agitator, drop funnel and reflux cooler, PT 100.

Execution: Components 1), 2), 3), 4) and 5) were weighted out one after the under while being stirred in the reaction vessel and heated to 91° C. reflux temperature. The component 6) was added continuously drop by drop through the drop funnel in 3.30 hours. Thereafter agitation was continued for 3 more hours at reflux temperature. The reaction rate control resulted in a product content of 24.1 % along with 32.9% educt. Thereafter the components 2) and 3) were again added in the same amount and heated for a further 4 hours at 90° C. in the reflux, in the mixture 34% product with 26.7% ethyl hexanal were then present.

Further Processing: After being cooled to room temperature the reaction mixture was washed neutral with water and ammonium chloride solution, dried with sodium sulphate and concentrated on the rotary evaporator.

397.6 g raw product was distilled in a Vigreux column of 20 cm. 92.9 g main product was obtained at boiling temperatures of 63-67° C./0.05 bar and a purity of 93.8% was obtained, as determined with a gas chromatograph.

Yield: 27.65 % of the theoretical

### Examples of Application

Formulation A: Mens Eau de Toilette: Perfume Oil

| Percent by wt. | Components |
|---|---|
| 0.3 | Liffaroma |
| 0.3 | Ethyl maltol (Veltol plus) |
| 0.5 | Geranyl acetate |
| 0.5 | Evernyl |
| 1.0 | Hexenol (beta gamma) |
| 1.0 | Norlimbanol |
| 1.5 | Cyclogalbanate |
| 2.0 | Spearmint oil Nat. F.W. |
| 3.0 | Allylamyl glycolate |
| 3.0 | Mugwort oil |
| 5.0 | Orange oil sweet Italian |
| 10.0 | Lemon oil Messina |
| 10.0 | Patchouli 83-2970 |
| 15.0 | Lavandin grosso |
| 25.0 | Dihydromyrcenol |
| 25.0 | Isoraldein 70 |
| 30.0 | Cumarin |
| 47.0 | Vanillin |
| 60.0 | Linalool |
| 5.0 | Galaxolide 50 DEP |
| 87.0 | Linalyl acetate |
| 100.0 | Fixolide NP |
| 140.0 | Hedione |
| 299.9 | Iso E super |
| 5.0 | Cyclovertal 1% in DPG |
| 5.0 | Cyclohexylsalicylate |
| 7.0 | Ambroxan |
| 10.0 | Troenan |
| 20.0 | Herbavert |
| 20.0 | Sandelice |
| 1.0 | 2-methyl-4-ethyl-2-octene-1-al (produced according to |
| | Example 1) as 1% solution in dipropylene glycol |

Evaluation as perfume: The presence of 2-methyl-4-ethyl-2-octene-1-al (I) caused in the above formulation an improved binding of the vanillin and the lemon cream note into the entire composition and this reminds the smeller of overheated and overfull beaches.

Formulation B: Lemon Base

| Percent by wt. | Components |
|---|---|
| 0.2 | Aldehyde C 7 |
| 2.0 | Aldehyde C 12 |
| 5.0 | Aldehyde C 9 |
| 4.0 | Verotyl |
| 7.0 | Geraniol pure |
| 10.0 | Aldehyde C 8 |
| 10.0 | Aldehyde C 10 |
| 10.0 | Linalool |
| 10.0 | Hedione |
| 20.0 | Terpinyl acetate pure |
| 20.0 | Citronellyl acetate |
| 30.0 | Terpineol |
| 40.0 | Citronellal |
| 50.0 | Geranyl acetate |
| 164.0 | Orange oil sweet |
| 183.3 | Lemon oil Messina |
| 350.0 | Citral AR |
| 2.0 | Cyclovertal |
| 5.0 | Peranat |
| 10.0 | Herbavert |
| 12.0 | Aldehyde 11-11 |
| 25.0 | Melusate |
| 30.0 | Cyclohexyl salicylate |
| 0.5 | 2-methyl-4-ethyl-2-octene-1-al |
| | (produced according to Example 1) |

Evaluation as perfume: The use of 2-methyl-4-ethyl-2-octene-1-al (I) caused in the composition the natural aroma of a freshly cut, juicy and sour lemon. A comparative composition which was structured like formulation B, but did not contain component (I) according to the invention, collapsed in contrast thereto into individual components which minded the smeller of the bitter aroma of crunched lemon seeds, fat, leather and walnuts.

1. 2-methyl-4-ethyl-2-octene-1-aldehyde of the structure (I)

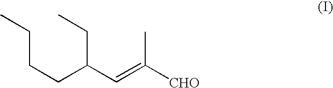

2. Method for the production of 2-methyl-4-ethyl-2-octene-1-aldehyde of the structure (I) 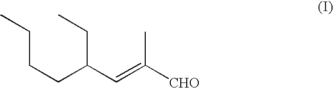
in which 2 ethyl hexanal is reacted with propion aldehyde in the presence of one or more organic compounds containing nitrogen as catalysts in an organic solvent.

3. Use as fragrance of 2-methyl-4-ethyl-2-octene-1-aldehyde of the structure (I)

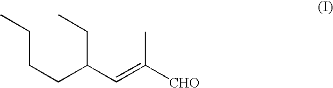

4. Fragrance compositions having a content of 2-methyl-4-ethyl-2-octene-1-aldehyde (I) according to claim 1, (I) being contained in the compositions in an amount of 1 to 70% by wt., based on the entire composition.
